(19) United States
(12) Patent Application Publication
(10) Pub. No.: US 2017/0300119 A1
(43) Pub. Date: Oct. 19, 2017

(54) INTRA-ORAL IMAGING USING OPERATOR INTERFACE WITH GESTURE RECOGNITION

(71) Applicant: Carestream Health, Inc., Rochester, NY (US)

(72) Inventors: Yingqian Wu, Shanghai (CN); Wei Wang, Shanghai (CN); Guijian Wang, Shanghai (CN); Yan Zhang, Shanghai (CN)

(21) Appl. No.: 15/315,002

(22) PCT Filed: Jun. 25, 2014
(86) PCT No.: PCT/CN2014/080732

§ 371 Date: Nov. 30, 2016

(51) Int. Cl. G06F 3/01; A61B 1/24; H04N 5/225; H04N 5/262; H04N 5/232; A61B 1/04; G06F 3/0482; H04N 5/217

(52) U.S. Cl. CPC G06F 3/017; H04N 5/23203; A61B 1/24; H04N 5/2256; H04N 5/2628; H04N 5/23293; H04N 5/23245; H04N 5/23216; A61B 1/04; G06F 3/0482; H04N 5/217; H04N 2005/2255

(57) ABSTRACT

A method for obtaining an intra-oral image, the method executed at least in part by a computer system, emits illumination from an intra-oral camera toward an object that is within the mouth of a patient, then obtains image data content of the object at an image sensor of the intra-oral camera. The image content obtained from the imaging sensor is displayed and one or more movement signals indicative of movement of the intra-oral camera along at least two of three mutually orthogonal axes is obtained. The one or more obtained movement signals are interpreted as an operator instruction corresponding to a predetermined movement pattern. At least the display of the image content is changed according to the operator instruction.

### FIELD OF THE INVENTION

The invention relates generally to the field of intra-oral imaging and more particularly relates to methods and apparatus for gesture-based operator interface interaction provided with the intra-oral camera.

### BACKGROUND OF THE INVENTION

Dental practitioners have recognized the value of intra-oral imaging apparatus for improving diagnostic capability, for maintaining more accurate patient records, and for improving their communication with patients. Advantages such as these, coupled with ongoing improvements in capability, compactness, affordability, and usability have made intra-oral imaging systems attractive for use in dental offices and clinics.

Using an intra-oral camera, a succession of digital images, such as video images, can be obtained from the mouth of the patient. As shown in FIG. 1A, the images obtained from an intra-oral camera 30 are generally displayed on a display monitor 20 that is visible to both a practitioner 16 and a patient 14. This capability allows practitioner 16 to more clearly visualize a problem area and can help to provide a better understanding of a recommended procedure for the patient.

A number of display features are available for the obtained digital images. Standard display functions, such as zooming in or out, panning, brightness or color adjustment, and other functions are readily available on a graphical user interface for improving the visibility of an affected area. In addition, the same display can be used as the operator interface for displaying or for entry of information about the patient, such as previous treatment or history data, patient identification, scheduling, and so on.

A practical problem that affects use of the display as an operator interface relates to command or instruction entry. If the practitioner is required to repeatedly switch between imaging functions and instruction or data entry, moving between the mouth of the patient and a keyboard or computer mouse, touch screen, or other data entry, selection, or pointing device, there is a potential risk of infection. Continually changing gloves or using replaceable covers or films for keyboard or touch screen and other devices are options; however, these can be impractical for reasons of usability, efficiency, likelihood of error, and cost. Often, a “four hands” solution is the only workable arrangement; for this, the dental practitioner enlists the assistance of another staff member for help with image view adjustment, patient data entry, and instruction entry during an imaging session.

There have been a number of solutions proposed for addressing this problem and allowing the practitioner to interact with the imaging system directly. These include, for example, the use of foot pedal control devices, voice sensing, infrared source tracking, and other mechanisms for instruction entry. Understandably, solutions such as these can be error-prone, can be difficult to calibrate or adjust, and can be awkward to set up and use.

Thus, there is a need for apparatus and methods that allow the dental practitioner to obtain images and enter patient data or instructions without requiring assistance from other members of the staff and without setting the intra-oral camera aside in order to change gloves.

### SUMMARY OF THE INVENTION

The present invention is directed to improvements in intra-oral image capture and display. Embodiments of the present invention address the problem of practitioner interaction with the intra-oral imaging apparatus for display of images and entry of instructions and patient data during an imaging session.

It is a feature of embodiments of the present invention that the intra-oral camera itself is configured to sense gestural instructions that are intended to send instructions that affect displayed information during the patient imaging session. Advantageously, embodiments of the present invention allow the dental practitioner to enter instructions for control of the display contents, entry of imaging parameters, or entry of patient data or other instructions using detected motion of the camera itself.

According to an embodiment of the present invention, there is provided a method for obtaining an intra-oral image, the method executed at least in part by a computer system and comprising: emitting illumination from an intra-oral camera toward an object that is within the mouth of a patient; obtaining image data content of the object at an image sensor of the intra-oral camera; displaying the image content obtained from the imaging sensor; obtaining one or more movement signals indicative of movement of the intra-oral camera along at least two of three mutually orthogonal axes; interpreting the one or more obtained movement signals as an operator instruction corresponding to a predetermined movement pattern; and changing at least the display of the image content according to the operator instruction.

According to another aspect of the present invention, there is provided an intra-oral imaging apparatus comprising: an intra-oral camera comprising: (i) a light source that is energizable to emit illumination toward an object that is within the mouth of a patient; (ii) an imaging sensor that is energizable to obtain image content of the object; (iii) a motion sensing element that provides one or more signals indicative of acceleration of the intra-oral camera along at least two of three mutually orthogonal axes; a display that displays obtained image content from the imaging sensor and that provides a graphical user interface for control of intra-oral camera imaging; a processor that is in signal communication with the motion sensing element and is configured to recognize an operator instruction according to the signals indicative of a predetermined movement pattern for the camera, detected by the motion sensing element; wherein the recognized operator instruction relates to the displayed image content for the patient and changes at least the graphical user interface on the display; and a switch that is in signal communication with the processor; wherein a switch position indicates to the processor either to acquire image content or to obtain an operator instruction.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1A is a perspective view showing use of an intra-oral camera for obtaining an image from the mouth of a patient.

FIG. 1B is a perspective view showing the use of an intra-oral camera for entering operator instructions in a command mode.

FIG. 2 is a schematic block diagram showing components of an intra-oral imaging system according to an embodiment of the present invention.

FIG. 3 is a perspective view that shows an intra-oral camera moved in different directions to provide user interface instructions.

FIG. 4 is a perspective view that shows three conventional Cartesian coordinate axes.

FIG. 5 is a graph showing accelerometer data measured over time for rotational movement.

FIG. 6 is a graph showing accelerometer data measured over time for linear movement.

FIG. 7 is a table showing a number of exemplary movement pattern vectors for instruction entry.

FIG. 8 is a logic flow diagram that shows a sequence for entry of an operator instruction when using the intra-oral camera in a command mode.

### DETAILED DESCRIPTION OF THE INVENTION

Figures provided herein are given in order to illustrate key principles of operation and component relationships along their respective optical paths according to the present invention and are not drawn with intent to show actual size or scale. Some exaggeration may be necessary in order to emphasize basic structural relationships or principles of operation. Some conventional components that would be needed for implementation of the described embodiments are not shown in the drawings in order to simplify description of the invention itself, including, for example, components that provide power and transmit data in a wired or wireless manner. In the drawings and text that follow, like components are designated with like reference numerals, and similar descriptions concerning components and arrangement or interaction of components already described are omitted.

In the context of the present disclosure, the terms “first”, “second”, and so on, do not necessarily denote any ordinal, sequential, or priority relation, but are simply used to more clearly distinguish one element or set of elements from another, unless specified otherwise.

In the context of the present disclosure, the term “energizable” describes a component or device that is enabled to perform a function upon receiving power and, optionally, upon receiving an enabling signal. An image sensor, for example, is energizable to record image data when it receives the necessary power and enablement signals.

In the context of the present disclosure, two elements are considered to be substantially orthogonal if their angular orientations differ from each other by 90 degrees+/−12 degrees.

In the context of the present disclosure, the term “actuable” has its conventional meaning, relating to a device or component that is capable of effecting an action in response to a stimulus, such as in response to an electrical signal, for example.

In the context of the present disclosure, the terms “user”, “viewer”, “technician”, “practitioner”, and “operator” are considered to be equivalent when referring to the person who operates the intra-oral imaging system, enters commands or instructions, and views its results. The term “instructions” is used to include entry of commands or of selections such as on-screen button selection, listings, hyperlinks, or menu selections. Instructions can relate to commands that initiate image capture, adjustments to or selections of imaging parameters or imaging process, such as still or video image capture or other selection of commands or parameters that control the functions and performance of an imaging apparatus, including commands that adjust the appearance of displayed features.

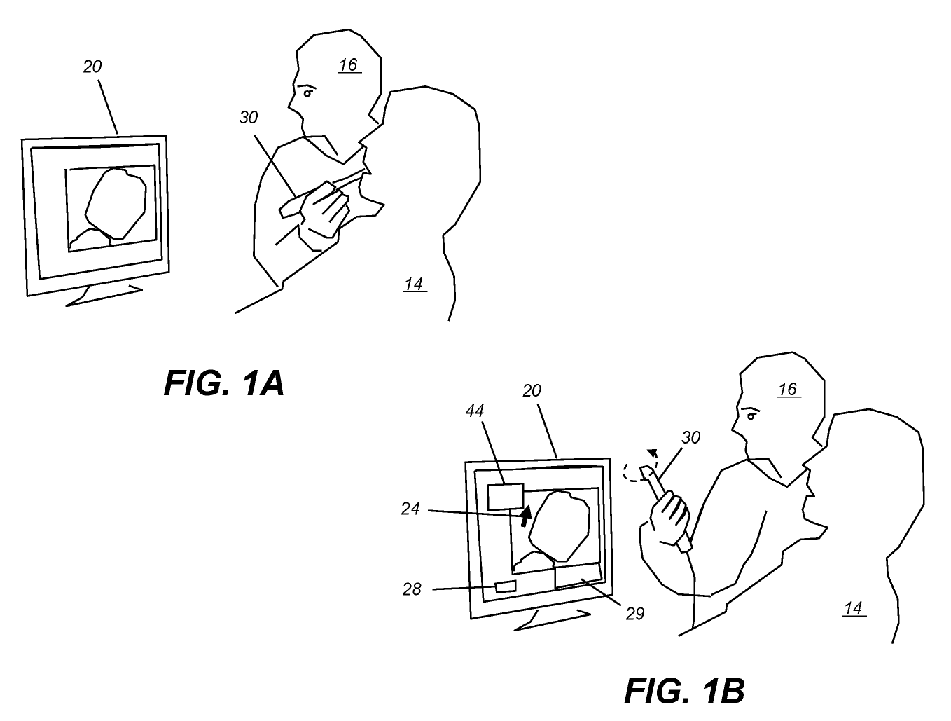

FIG. 1A showed dental practitioner 16 obtaining an image from the mouth of patient 14 using intra-oral camera 30 and viewing results on display 20. Entry of instructions, such as those needed to pan, zoom, or otherwise adjust what appears on display 20, is difficult for practitioner 16 without either a staff assistant or using some type of hands-free input device. FIG. 1B shows the practitioner 16 using intra-oral camera 30 in a command mode for instruction entry, according to an embodiment of the present invention. By making any of a number of predetermined gestures with intra-oral camera 30, such as briefly moving the camera 30 in a horizontal direction from left to right or moving the camera in a circular motion about an axis as shown in FIG. 1B, the practitioner 16 can enter instructions to perform functions such as pan or zoom of the display; brightness, color, contrast, or other image quality adjustment; display and selection from a pull-down menu 44 or control button 28; on-screen cursor 24 positioning; or data entry, such as from an on-screen keypad 29.

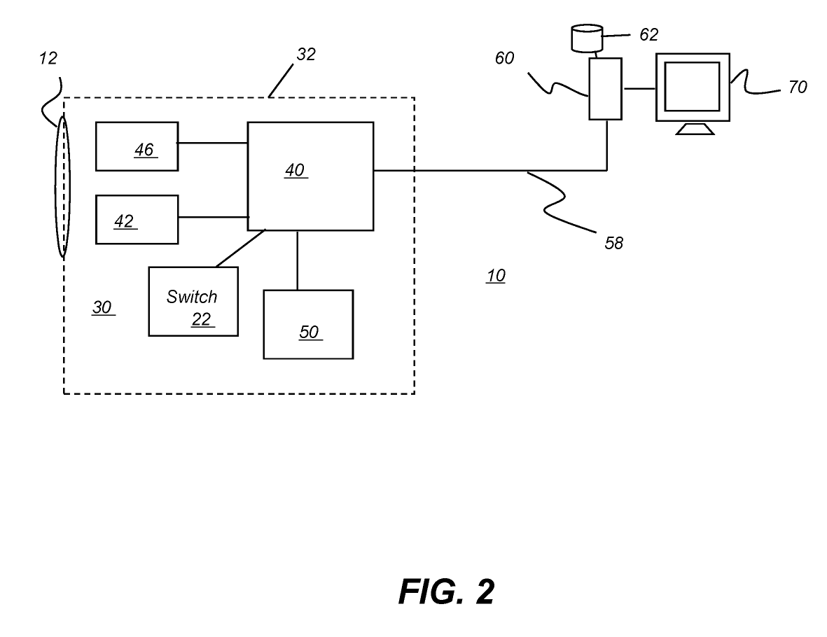

Consistent with an embodiment of the present invention, the schematic block diagram of FIG. 2 shows an intra-oral imaging apparatus 10 for obtaining an image of one or more objects, such as teeth, in the mouth of the patient. The components housed within a chassis 32 of an intra-oral camera 30 are shown within a dashed outline. A light source 46 provides illumination to the object, such as single color or white light illumination or infrared (IR) or ultraviolet (UV) light, or a combination of light having different spectral content. Light source 46 is in signal communication with and controlled by one or more signals from a processor 40. Optics 12, such as one or more lens elements, filters, polarizers, and other components, condition and direct the imaged light to an image sensor 42, such as a CCD (Charge-Coupled Device) array or CMOS (Complementary Metal-Oxide Semiconductor) array, which is in signal communication with processor 40 and provides image data to processor 40. An optional switch 22 is provided for manually switching camera 30 from a command mode into an imaging mode. It should be noted that the function of switch 22 for switching between camera modes can alternately be executed automatically by interpreting detected camera 30 motion, since pre-determined movements of camera 30 that are used for instruction entry, as described subsequently, are different from movement patterns typically used during image capture. The function of switch 22 can also be executed by determining camera 30 focus. According to an embodiment of the present invention, command mode is disabled during imaging, either for single images or for video imaging. According to an alternate embodiment of the present invention, keyboard or mouse command entry at the graphical user interface of display 70 (FIG. 2) overrides command entry from movement patterns.

In the FIG. 2 embodiment, a motion sensing element 50, such as a 3-D accelerometer or a set of multiple accelerometers, provides motion information that is used for user interface instruction entry. Processor 40 uses this information in order to detect operator instructions, as described in more detail subsequently. Processor 40 is a control logic processor that obtains the image data from image sensor 42 and instruction-related motion operation from motion sensing element 50 and provides this data for display. Processor 40 is in signal communication with a host computer or other processor 60 over a communication link 58, which may be a wired or wireless communication link. Host computer 60 is in signal communication with a display 70 for display of the obtained image and patient information and for entry of user interface instructions. Display 70 provides a graphical user interface for controlling and using intra-oral camera 30. According to an embodiment of the present invention, the graphical user interface on display 70 displays the command that has been entered using camera 30 movement. In addition, commands entered according to spatial movement patterns of camera 30 change the display of acquired image content on the graphical user interface. Host computer 60 is also in signal communication with a memory 62 for short- or long-term storage of patient image data and related patient information, such as treatment history and personal information about the patient.

Still considering the arrangement of FIG. 2, it can be appreciated that the functions described for processor 40 and host computer 60 can be performed by a more powerful processor 40 on intra-oral camera 30 itself, thus eliminating the need for the external host computer 60. Memory 62 can also be provided to processor 40, stored on camera 30. Processor 40 may also connect directly to display 70 for display of image content obtained from image sensor 42. Alternately, processor 40 can have only a data conditioning function and be primarily a transmitter device that simply provides all of its acquired data to host computer 60 for more complex image processing and motion analysis functions for recognizing operator commands. It can be appreciated that compact packaging of intra-oral camera 30 may dictate how much processing and storage capability can be provided within the body of camera 30.

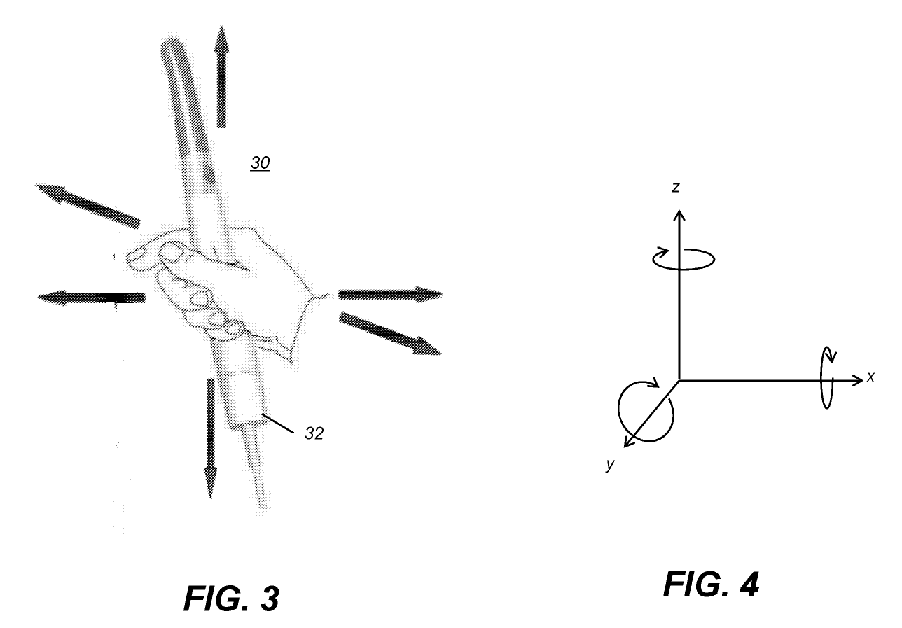

FIG. 3 shows the hand-held intra-oral camera 30 used for user interface instruction entry in a command mode. Arrows indicate some of the possible motion that can be provided for entering commands. FIG. 4 shows the three orthogonal axes for 3-dimensional (3-D) movement, conventionally known as Cartesian coordinate axes and identified as x, y, and z axes, respectively. Accelerometers used for motion sensing element 50 can measure movement in space with respect to any of the x, y, and z axes, as well as rotation relative to the axes, as shown.

Accelerometers can be micro-electromechanical system (MEMS) devices, such as those conventionally used in various types of smart phone and handheld personal computer pads and similar devices. The accelerometer output is a movement signal that is indicative of static acceleration, such as due to gravity, and dynamic acceleration from hand and arm movement of the operator and from other movement, such as from hand vibration. Since there is always some inherent noise in the accelerometer output, the measured activity from the movement signal is generally non-zero.

According to an embodiment of the present invention, a single 3-D accelerometer is used to detect motion along any of the three coordinate axes of FIG. 4. According to an alternate embodiment of the present invention, three accelerometers are used, each sensing motion along a corresponding one of the three orthogonal axes, respectively, as shown in FIG. 4. It can be appreciated that one, two or three accelerometers can alternately be used, in various configurations, for various levels of measurement range and accuracy, each accelerometer providing a corresponding movement signal for interpretation by processor 40 (FIG. 2).

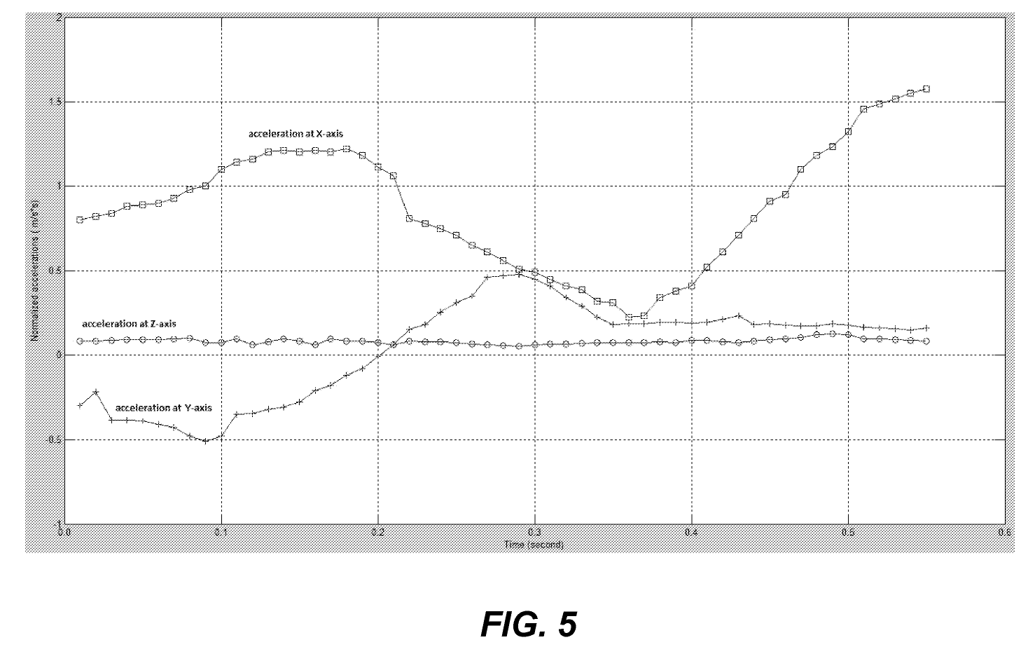

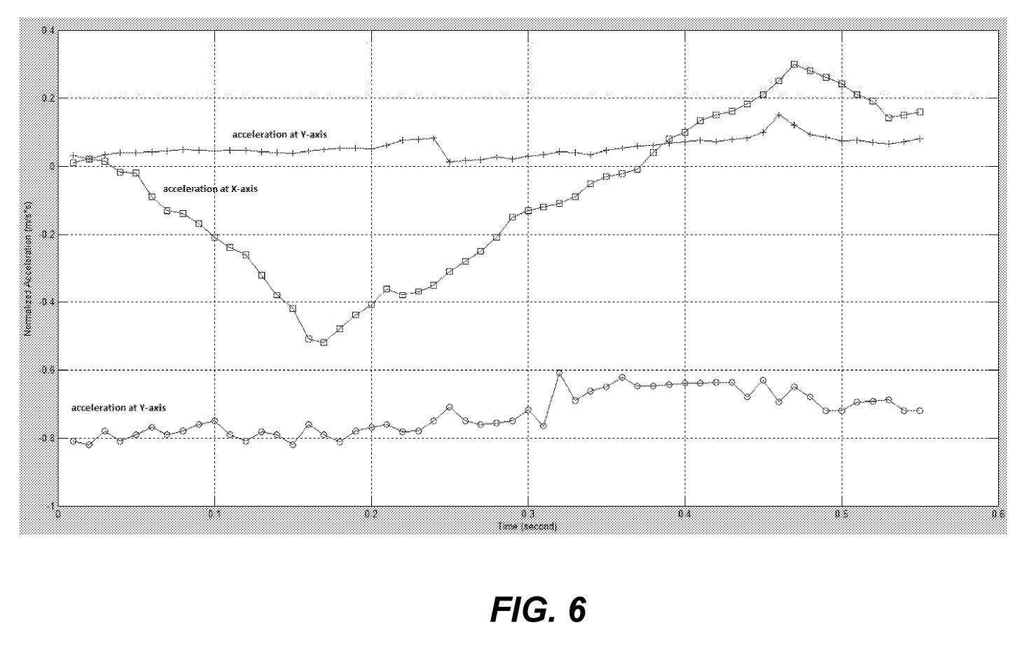

FIGS. 5 and 6 show characteristic curves obtained from sampling the movement signal data from motion sensing element 50 when using multiple accelerometers. FIG. 5 shows normalized accelerometer data collected, over time, when the intra-oral camera 30 is moved rotationally in a clockwise (CW) circle. FIG. 6 shows normalized accelerometer data collected, over time, when the intra-oral camera 30 is moved horizontally along a line from left (L) to right (R). The acceleration scale is normalized relative to gravity. These characteristic curves provide sufficient information for identifying the movement path and duration and are interpreted for entry of various user interface instructions according to movement of intra-oral camera 30 in command mode.

As noted earlier, intra-oral camera 30 can be in an imaging mode or in a command mode, according to the position of optional switch 22 (FIG. 2). It can be appreciated that mode selection can alternately be performed in an automated manner, such as by sensing whether or not camera 30 is focused on a tooth or other object or is removed from the mouth and held in a position from which no object is in focus. Methods of focus detection that can be used for this type of automatic mode determination are well known to those skilled in the imaging arts. Still other methods of determining the mode of intra-oral camera 30 relate to detecting movement of camera 30 as reported by motion sensing element 50. The predetermined movement patterns of camera 30 that are used to enter instructions are generally executed at speeds that would cause significant amounts of blur in obtained images.

An alternate source for movement sensing relates to image blur. According to an alternate embodiment of the present invention, the camera 30 mode, for imaging mode or command mode, is determined using a combination of both acceleration data and focus detection. Image analysis detects camera 30 motion and provides a movement signal that is indicative of accelerometer data and, optionally, image analysis.

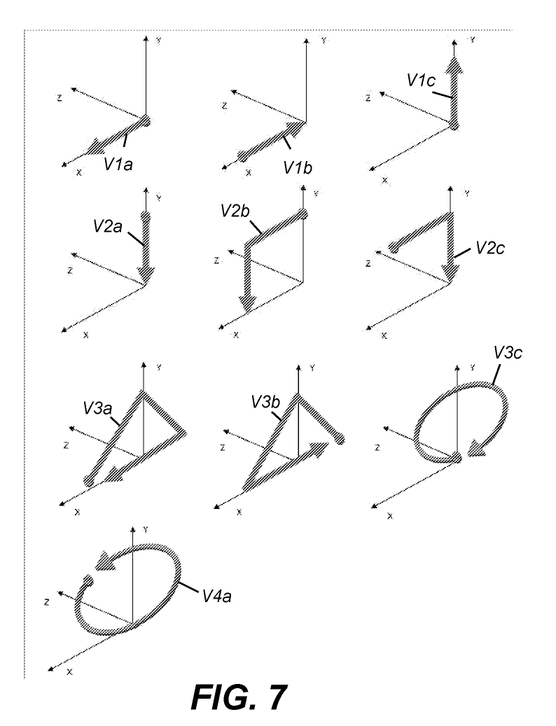

FIG. 7 shows a table that lists, by way of example and not by way of limitation, some of the characteristic movement patterns of camera 30 that can be readily detected by the one or more accelerometers used in motion sensing element 50, consistent with an embodiment of the present invention. Each block of the table shows a movement pattern with its movement pattern vector and shows the corresponding orthogonal axes over which movement can be sensed. A dot in each block represents the beginning of a movement pattern; the arrow shows movement direction. A vector V1a shows a left-to-right (L-R) movement pattern, measured relative to x and y axes. A vector V1b shows the opposite right-to-left (R-L) movement pattern, measured relative to x and y axes. A vector V1c shows a vertical movement pattern in the upward direction, measured relative to x and y axes. A vector V2a shows a vertical movement pattern in the downward direction, measured relative to x and y axes. Vectors V2b and V2c show right angle movement patterns, relative to x-y axes. Vectors V3a and V3b show triangular movement patterns, relative to x-y axes. Vectors V3c and V4a show circular movement patterns in different clockwise (CW) and counter-clockwise (CCW) directions, measured relative to the indicated x-z and y-z axes. In addition to those patterns shown, other movement patterns, such as “w” shaped movement patterns, relative to two or more axes, can be used. Each of these characteristic movement patterns can be detected using the arrangement of one, two, or three accelerometers for motion sensing element 50 as described previously with reference to FIG. 2. Each of these and other movement patterns can be used to provide a movement signal that indicates an operator instruction. As noted previously, image processing can also be used to supplement or to verify or validate movement data from motion sensing element 50 according to detection of motion blur.

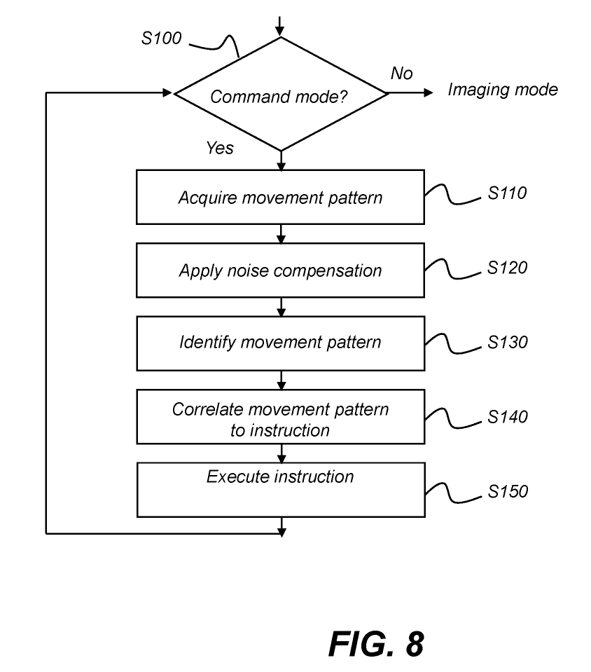

Consistent with an embodiment of the present invention, the logic flow diagram of FIG. 8 shows a sequence of steps used for executing user interface instructions according to detected movement in command mode. This sequence of steps runs continuously when in command mode. In a mode decision step S100, the mode of operation of intra-oral camera 30 is detected as either imaging mode or command mode. For the camera embodiment shown in FIG. 2, switch 22 indicates the camera mode as either command mode or imaging mode. If in imaging mode, movement of the intra-oral camera 30 is not interpreted for command entry. If the camera 30 is in command mode, then a pattern acquisition step S110 executes, acquiring the measurement data from motion sensing element 50. Where motion sensing element 50 is a single 3-D accelerometer, for example, a time series of 3-dimensional vector data acquired by the accelerometer is provided as input to the gesture detection steps shown here. A number of measurements are obtained for characterizing the movement pattern, as was described previously with reference to the graphs of FIGS. 5 and 6. An optional noise compensation step S120 eliminates noise data from random movement, such as unintended or incidental movement along or about one or possibly two of the orthogonal axes. This noise removal is useful because the practitioner is not likely to move the camera 30 in precisely one direction or to provide rotation that is symmetrical about a single axis, for example. Vibration from the dental office environment or from nearby equipment can also add some noise content. With respect to the example of FIG. 6, for example, movement along the y and z directions appears to be unintended, whereas movement along the x axis appears to be intentional and is prominent.

Continuing with the sequence shown in FIG. 8, a pattern identification step S130 can then be executed, identifying the most likely movement pattern indicated by the measured data, such as that shown in the table of FIG. 7. Once the most likely pattern is identified, an instruction identification step S140 then correlates the movement pattern to a corresponding instruction. An instruction execution step S150 then executes the entered instruction. The sequence continues for additional command entry, looping back to mode decision step S100 as shown. Ambiguous movement data is possible and the practitioner observes the display 20 screen to ascertain that the intended instruction has been received. In some cases, a prompt or other verification is posted to the display screen, requesting clarification or verification of the entered command. This can also be provided, for example, with a movement pattern that is not likely to be unambiguous, such as by the movement pattern shown by vector V1c or V2a in FIG. 7, for example.

Consistent with an embodiment of the present invention, a standard set of predetermined movement patterns for intra-oral camera 30 is provided, with each pattern identifying a unique, corresponding instruction for operator entry, such as the set of movement patterns shown in FIG. 7. A default set of movement characteristics is initially used. However, motion sensing element 50 can further be trained to identify additional instructions or trained to respond to a particular set of instructions from the practitioner. Gesture training software, optionally provided as part of intra-oral imaging system 10 (FIG. 2) uses many of the same components that are employed for gesture detection. According to an embodiment of the present invention, software containing training algorithms for resetting and calibrating gestures with intra-oral camera 30 are provided as part of processor 40 (FIG. 2).

According to an embodiment of the present invention, the practitioner has a setup utility that allows redefinition of one or more movement patterns as well as allowing additional movement patterns to be defined and correlated with particular operator instructions. This utility can be particularly useful for customizing how the imaging system performs various functions. As just one example, a zoom-in viewing function may be customized to zoom in fixed, discrete increments, such as at 100%, 150%, and 200%, with an increment change effected with each completion of a movement pattern. Alternately, zoom-in can be continuous, so that zoom operation continuously enlarges the imaged object as long as the operator continues the corresponding movement pattern. In addition, the setup utility can also be used to adjust sensitivity and sampling rate of motion sensing element 50 to suit the preferences of the dental practitioner who uses intra-oral imaging system 10.

Motion sensing element 50 can use any suitable number of accelerometers or other devices for measuring motion along orthogonal axes. Options for motion sensing element 50 include the use of only one or two accelerometers, or the use of three or more accelerometers for measuring movement in appropriate directions. MEMS accelerometer devices are advantaged for size, availability, and cost; other accelerometer types can alternately be used. Alternately, gyroscopes, magnetometers, and other devices that are capable of measuring measure movement along an axis or rotation about an axis can be used.

Consistent with an embodiment of the present invention, a host processor or computer executes a program with stored instructions that provide imaging functions and instruction sensing functions in accordance with the method described. As can be appreciated by those skilled in the image processing arts, a computer program of an embodiment of the present invention can be utilized by a suitable, general-purpose computer system, such as a personal computer or workstation. However, many other types of computer systems can be used to execute the computer program of the present invention, including networked processors. The computer program for performing the method of the present invention may be stored in a computer readable storage medium. This medium may comprise, for example; magnetic storage media such as a magnetic disk (such as a hard drive) or magnetic tape or other portable type of magnetic disk; optical storage media such as an optical disc, optical tape, or machine readable bar code; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other communication medium. Those skilled in the art will readily recognize that the equivalent of such a computer program product may also be constructed in hardware.

It will be understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

The invention has been described in detail with particular reference to a presently preferred embodiment, but it will be understood that variations and modifications can be effected within the spirit and scope of the invention. For example, the function of optional switch 22 (FIG. 2) for changing the intra-oral camera 30 between imaging and command modes can be effected by sensing, so that the camera 30 is automatically placed in command mode when movement or imaging data indicate that the camera 30 is not in the patient's mouth. Various movement patterns can be provided in addition to the examples shown in FIG. 7. The image processing and operation logic functions described with reference to FIG. 2 can be performed on a single processor that resides internally on intra-oral camera 30, on an external host processor 60, or on some combination of internal and external logic processing devices, including one or more networked computers, for example. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for obtaining an intra-oral image, comprising:
emitting illumination from an intra-oral camera toward an object within the mouth of a patient;
obtaining image data content of the object at an image sensor of the intra-oral camera;
displaying the image content obtained from the imaging sensor;
obtaining one or more movement signals indicative of movement of the intra-oral camera along at least two of three mutually orthogonal axes;
interpreting the one or more obtained movement signals as an operator instruction corresponding to a predetermined movement pattern; and
changing at least the display of the image content according to the operator instruction.

2. The method of claim 1 further comprising changing one or more imaging parameters according to the one or more obtained movement signals.

3. The method of claim 1 further comprising providing an operator interface that is responsive to the one or more obtained movement signals.

4. The method of claim 1 wherein changing the display of the image content changes the zoom magnification of the displayed image content.

5. The method of claim 1 wherein changing the display of the image content changes the panning of the displayed image content.

6. The method of claim 1 further comprising indicating a menu selection on the display according to the operator instruction.

7. The method of claim 1 wherein interpreting the one or more obtained signals further comprises using results from training software.

8. The method of claim 1 wherein obtaining the one or more signals indicative of movement of the intra-oral camera further comprises sensing a switch position.

9. An intra-oral imaging apparatus comprising:
an intra-oral camera comprising:
(i) a light source that is energizable to emit illumination toward an object that is within the mouth of a patient;
(ii) an imaging sensor that is energizable to obtain image content of the object; and
(iii) a motion sensing element that provides one or more signals indicative of acceleration of the intra-oral camera along at least two of three mutually orthogonal axes;
a display that displays obtained image content from the imaging sensor and that provides a graphical user interface for control of intra-oral camera imaging;
a processor in signal communication with the motion sensing element and configured to recognize an operator instruction according to the signals indicative of a predetermined movement pattern for the camera, detected by the motion sensing element, the recognized operator instruction relating to the displayed image content for the patient and changes at least the graphical user interface on the display; and
a switch that is in signal communication with the processor, wherein a switch position indicates to the processor either to acquire image content or to obtain an operator instruction.

10. The intra-oral imaging apparatus of claim 9 wherein the motion sensing element comprises one or more accelerometers.

11. The intra-oral imaging apparatus of claim 9 wherein the motion sensing element comprises a gyroscope or a magnetometer.

12. The intra-oral imaging apparatus of claim 9 wherein the processor is within a chassis of the intra-oral camera.

13. The intra-oral imaging apparatus of claim 9 wherein the operator instruction from the movement pattern appears on the graphical user interface of the display.

14. The intra-oral imaging apparatus of claim 9 wherein the operator instruction performs a pan or zoom adjustment of the displayed image content.

15. The intra-oral imaging apparatus of claim 12 wherein the processor is in signal communication with the display.

16. The intra-oral imaging apparatus of claim 9 wherein the intra-oral camera provides a signal that indicates whether it is in an imaging mode, within the patient's mouth, or in a command mode, outside the patient's mouth.

17. The intra-oral imaging apparatus of claim 12 wherein the processor is a first processor and is within the chassis of the intra-oral camera and further comprising a second processor that is in signal communication with the first processor and wherein the second processor is further in signal communication with the display.

18. The intra-oral imaging apparatus of claim 11 wherein the signal communication between the first and second processor is wireless communication.

19. The intra-oral imaging apparatus of claim 16 wherein the intra-oral camera detects motion blur to determine whether it is in the imaging or the command mode.
